(19) United States
(12) Patent Application Publication
(10) Pub. No.: US 2005/0043809 A1
(43) Pub. Date: Feb. 24, 2005

(54) Osteoprosthesis component

(76) Inventor: Leif Ryd, Linkoping (SE)

Correspondence Address: Paul J Maginot;Maginot Moore & Beck, Bank One Center/Tower, 111 Monument Circle, Suite 3000, Indianapolis, IN, 46204-5115, US

(21) Appl. No.: 10/490,270

(22) PCT Filed: Sep. 20, 2002
(86) PCT No.: PCT/GB02/04264

(30) Foreign Application Priority Data
Sep. 21, 2001 (GB) 0122779.2

(51) Int. Cl. A61F 2/38; A61F 2/30

(52) U.S. Cl. 623/20.32; 623/18.11

(57) ABSTRACT

An osteoprosthesis component which is suitable for fitting to the end of a first bone which has been resected, in a joint between that bone and a second bone, includes a transverse portion which can be fitted across the resected end of the first bone. The transverse portion has a planar first surface which faces the resected end of the first bone when the component is implanted and a second surface for engaging the second bone directly or indirectly. The component includes a rail portion around at least part of the periphery of the component extending in a direction away from the second bone, in which the inwardly facing surface of the rail portion is generally rounded extending continuously from the planar first surface of the transverse portion when the component is viewed in cross-section.

This invention relates to an osteoprosthesis component of the kind which is fitted onto the resected end of a bone at a joint between that bone and another bone.

Components of artificial joints, for example a hip, elbow or shoulder joint, and particularly a knee joint, are commonly fixed to natural bone tissue after the end of the bone has been removed. This enables original bone tissue that is damaged and which contacts another bone in a joint to be replaced so that discomfort which results from joint articulation can be reduced. An artificial joint component to be fitted to the resected bone will often comprise a transverse portion which engages and is supported against axial load at or towards its edge by cortical bone tissue the resected end of the bone. The other bone can then act (directly or indirectly) against the transverse portion of the component.

The transverse portion can have one or more pegs depending from it, which extend into the intramedullary cavity within the bone. The peg(s) can help to locate the component transversely relative to the longitudinal axis of the bone. A peg will often be tapered which can facilitate contact between the external surface of the peg and the internal surface of the intramedullary cavity. It can sometimes be preferred for a bond to be formed between the peg and bone tissue within the cavity, contributing to the prevention of movement of the artificial joint component relative to the bone tissue.

It has been found that location of a central peg in the intramedullary cavity of a bone might not always eliminate satisfactorily movement of the prosthesis component relative to the end of the resected bone. Furthermore, removal of the end of a bone for implanting a prosthetic joint component has the disadvantage that the structure of the natural bone tissue is weakened, in particular when exposed to forces directed along the axis of the bone. Such forces tend to compress bone tissue axially, and the forces are then transmitted transversely onto the cortical bone tissue.

The present invention provides an osteoprosthesis component which includes a transverse portion which can be fitted across the resected end of a bone, and a rail portion around at least part of the periphery of the component, in which the inwardly facing surface of the rail portion is generally rounded extending continuously from the planar surface of the transverse portion when the component is viewed in cross-section.

Accordingly, in one aspect, the invention provides an osteoprosthesis component which is suitable for fitting to the end of a first bone which has been resected, in a joint between that bone and a second bone, which comprises a transverse portion which can be fitted across the resected end of the first bone, having a first surface for contacting the resected end of the first bone and a second surface for engaging the second bone directly or indirectly, in which the first surface is generally planar, and a rail portion around at least part of the periphery of the component extending in a direction away from the second bone, in which the inwardly facing surface of the rail portion is generally rounded extending continuously from the planar surface of the transverse portion when the component is viewed in cross-section.

The rail portion can present a substantially continuous surface directed towards the axis of the bone to support the bone tissue against outwardly directed transverse forces to which the component is exposed when the joint is under load. Those might otherwise lead to transverse movement of the prosthesis component relative to the end of the resected bone. The resulting hoop stresses might also cause damage to the cortical bone tissue.

The prosthesis of the present invention has the advantage that, when an axial load is applied to the bone through the prosthesis, the natural bone tissue is better able to accommodate the outwardly directed transverse forces which can give rise to hoop stresses. The prosthesis therefore has an advantageous combination of formations which help to locate it relative to the natural bone tissue (and optionally to contribute to fixing it to that tissue), while also helping the tissue to withstand adverse biomechanical forces to which it will be exposed by the prosthesis when in use.

Preferably, the ratio of the distance from the top of the rail to the point at which the rail meets the planar surface of the transverse portion (measured parallel to the planar surface) to the height of the rail above the planar surface, measured normal to the planar surface, is at least about 1.5, preferably at least about 1.75, more preferably at least about 2.0. Particularly beneficial results are obtained when the value of the ratio is 3.0 or more The value of the ratio will generally be less than about 20, preferably less than about 15, more preferably less than about 10, for example less than about 8.

Preferably, the angle between the tangent to the inwardly facing surface of the rail portion at the top of the rail and the normal to the planar surface of the transverse portion which passes through the top of the rail is at least about 300, preferably at least about 40° or 45°, more preferably at least about 50°, for example at least about 65°. The angle will generally be less than about 90°, preferably less than about 80°. It has been found that acceptably low levels of micromotion of the prosthesis component relative to the underlying resected bone can be obtained by maintaining the said angle above 30° or 40°.

The inwardly facing surface of the rail portion (which is the surface of the rail which faces towards the bone axis) can have a circular shape when the component is viewed in cross section so that, in a component of which at least a part is circular when the component is viewed in plan, the surface will be generally toroidal. Generally however it will be preferred for the inwardly facing surface of the rail portion to have a rounded but non-circular shape when the component is viewed in cross-section. The shape will generally be such that the radius curvature of the surface (when the component is viewed in cross-section) decreases from a maximum at the point at which the inwardly facing surface of the rail portion meets the planar surface of the transverse portion. Preferably, the radius of curvature decreases continuously and monotonically from the point at which the inwardly facing surface of the rail portion meets the planar surface of the transverse portion towards the top of the rail. Examples of surface shapes which might be used in the present invention include shapes defined by a part of any of an ellipse, an involute and a parabola. A shape defined by a part of a parabola is particularly preferred.

When the radius of curvature of the surface is large, especially around the region in which the rail portion meets the planar surface of the transverse portion, the first surface of the transverse portion might only be small, and the surface which contacts the first bone might then be provided largely by the curved inwardly facing surface of the rail portion.

It has been found that the forces that are required to minimise lateral motion between a resected bone and a prosthesis component according to the present invention can be optimised by configuring the component according to the parameters referred to above.

Preferably, the rail portion of the component is tapered towards its top, so that it is wider at its root than at its top. This can be achieved conveniently by arranging the outwardly facing surface of the rail portion so that it extends approximately normal to the planar portion of the first surface. The rail portion can be tapered over virtually its entire height. This can optimise the inwardly directed forces to which the bone is exposed due to contact between the bone and the rail portion. Alternatively, the rail portion might be tapered along just part of its height. For example, tapering the rail portion in the top region can facilitate penetration of the rail portion into natural bone tissue, during or after implantation of the prosthesis, or both.

The rail portion might be differently tapered at different regions around the periphery of the component: for example, it might be tapered towards its top in one region, and not tapered in another region (for example, with a concave outwardly facing surface).

The height of the rail portion (measured from the planar first surface of the transverse portion) will be selected according to factors such as the nature and configuration of the bone which it is intended to contact and the nature of the forces which are generated in the bone when the joint is placed under load. The height of the rail portion can be uniform around the component. It might however be preferable to arrange the rail portion with a height which differs in one region of the component from another region. Generally, the height of the support formation will be at least about 1.5 mm, preferably at least about 2.0 mm, for example at least about 3.0 mm. The height will generally be not more than about 15 mm, preferably not more than about 10 mm, especially not more than about 8 mm.

Preferably, the rail portion extends around at least about 60% of the periphery of the component, more preferably at least about 75%, more preferably at least about 90%.

The component can be configured to be fitted to the tibia in a total knee replacement. The rail portion can then be provided on at least the medial and lateral edges of the component. Preferably it will also be provided on the anterior edge as well, and in some cases the posterior edge. When the rail portion is not provided at a portion of the edge of the component, the planar surface of the transverse portion of the first surface can extend to the edge of the component, for example in at least an approximately central part of the posterior edge, especially in the region of a notch. Preferably, the rail portion extends continuously around the anterior, medial and lateral edges of a tibial component of a knee prosthesis, and around the medial and lateral ends of the posterior edge.

Preferably, the component includes at least one peg which depends from the transverse portion so that it can extend into the intramedullary cavity of the bone when the component is in contact with the resected end of the bone with its first surface facing the bone.

Preferably, the peg (or pegs when there are more than one) is configured to optimise the contact between it and the internal surface of the cavity (generally the intramedullary cavity) of a bone in which it is to be inserted when in use. For example, the peg is preferably tapered so that there is contact between the peg and the internal surface of the cavity so that the prosthesis is located transversely relative to the axis of the bone.

When there is just one peg, it will be located generally centrally on the first surface of the transverse portion of the prosthesis, so that it can be received in the intramedullary cavity of the bone with the first surface of the transverse portion facing the resected end of the bone.

The surface of the component which is intended to contact the bone can be configured to optimise the formation of a bond between it and the bone tissue. This can include the surface of the peg (when present). For example, when the component is to be used for cementless applications, the surface of the peg can be made porous to encourage the ingrowth of bone tissue. When the component is to be fixed to the bone using bone cement, the component can be configured to accommodate a layer of bone cement between it and the bone.

The prosthesis of the invention will be formed from a material which has suitable biocompatibility. Metallic materials, especially alloys such as certain stainless steels and alloys based on titanium, and cobalt-chrome can be used. The prosthesis can be formed from or coated with non-metallic materials, such as ceramic materials. The prosthesis can be formed by conventional forming techniques such as casting, forging, welding and so on.

The prosthesis of the invention can be used with advantage in joints in which a peg (especially a tapered peg) is located within the intramedullary cavity of a bone to locate a prosthesis transversely relative to the axis of the bone. The component can be fixed to the bone using bone cement or it can be used in cementless applications. The invention finds particular application in the tibial component of a knee joint prosthesis when the first surface of the transverse portion can contact a femoral component, generally indirectly through an intermediate bearing component. However, its use in the tibial component of a knee joint includes use without a peg. It can also be used as the femoral component in a hip joint or the humeral component in a shoulder joint, when the transverse portion comprises a collar extending around the stem portion which is intended to extend into the intramedullary cavity of the bone.

Embodiments of the present invention will now be described by way of example with reference to the accompanying drawings, in which:

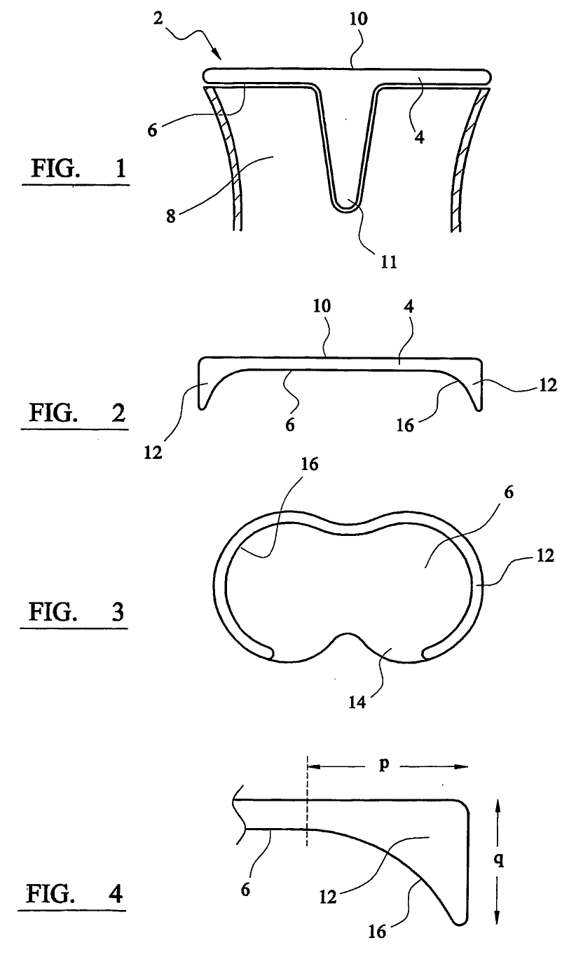

FIG. 1 is a side view in cross-section of a conventional tibial component of a knee joint prosthesis.

FIG. 2 is a side view in cross-section of a tibial component of a knee joint prosthesis in accordance with the present invention.

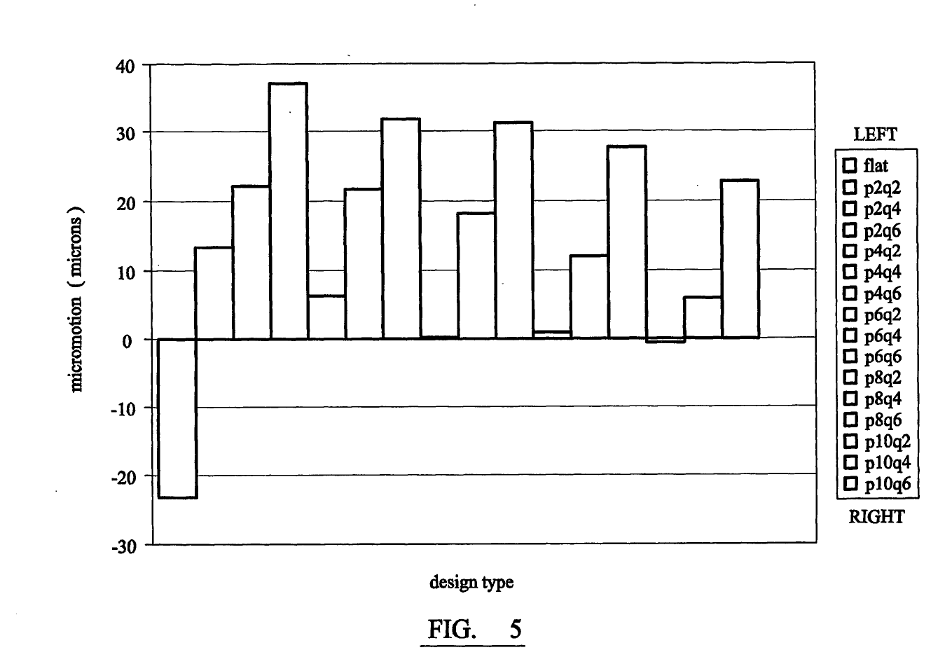

FIG. 3 is a view from below of the tibial component shown in FIG. 2.

FIG. 4 is an enlarged view of the edge region of the tibial component shown in FIGS. 2 and 3.

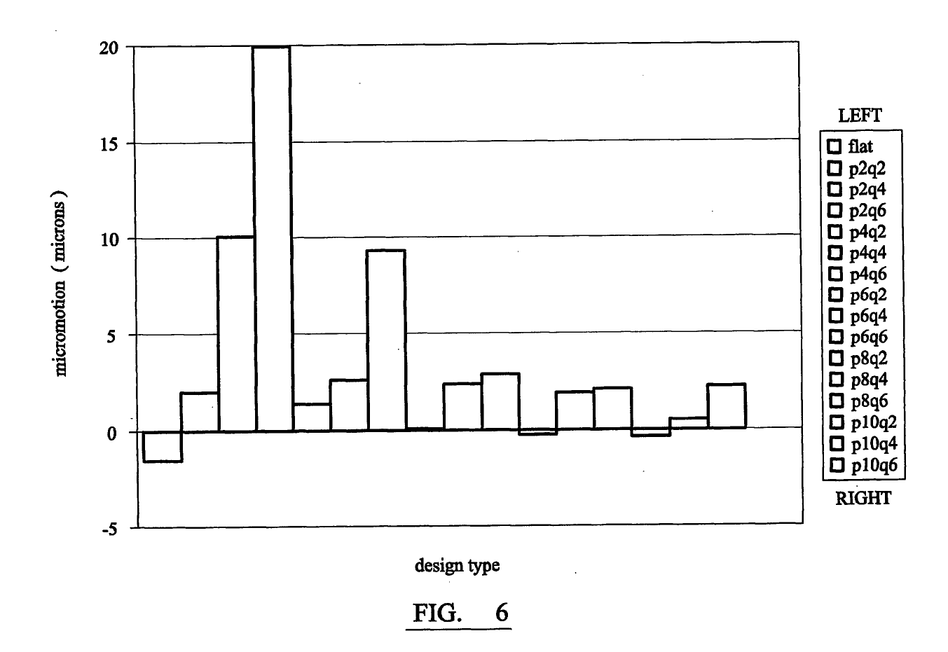

FIGS. 5 and 6 are graphs showing the degree of micromotion of tibial components having a range of configurations, with friction coefficients between the bone and the component of 0.1 and 0.7 respectively.

Referring to the drawings, FIG. 1 shows the tibial component 2 of a knee joint prosthesis. It includes a transverse portion 4 having a first surface 6 which contacts the resected end of the tibia 8 and a second surface 10 which faces the femoral component. A meniscus component (not shown) is generally positioned between the tibial and femoral components so that the femoral component engages the tibial component indirectly, through the meniscus component.

On its first surface 6, the tibial component has a central peg 11 which penetrates the intra-medullary cavity in the tibia when the first surface is positioned against the end of the resected bone. The peg is tapered so that there is contact between the peg and the internal surface of the cavity so that the prosthesis is located transversely relative to the axis of the bone. The outer surface of the peg can be porous to encourage the ingrowth of bone tissue to aid fixation of the component relative to the patient's bone tissue. However, transverse fixation of the component by location of the peg in the intramedullary cavity is not always satisfactory and there can be small amounts of transverse movement between the tibia and the prosthesis component. Such movement can lead to loosening of the component and failure of the prosthesis.

Apart from the central peg 11, the first surface 6 of the tibial component shown in FIG. 1 is planar. When the component is placed under an axial load, hoop stresses are generated in the cortical bone tissue of the tibia. High hoop stresses can lead to damage to the cortical bone tissue.

The prosthesis component of the present invention is shown in FIGS. 2 and 3, using the same reference numerals as used in FIG. 1. The tibial component has a rail 12 at its edge, around the planar first surface 6 of the transverse portion 4. The rail is provided around the edge of the component on its first surface, with the exception of the central region 14 of the posterior edge where the component has a notch (see FIG. 3). The inwardly facing surface 16 of the rail is rounded when the component is viewed in cross-section. The rounded inwardly facing surface extends continuously from the planar first surface so that there is no significant discontinuity on the surface. The rounded inwardly facing surface has a parabolic shape (when the component is viewed in cross-section as in FIG. 2) with the origin of the parabola at the point where the surface joins the planar surface of the transverse portion, so that the radius of curvature of the surface decreases continuously and monotonically from that point towards the top of the rail. The shape of the component might however have another shape such as that of a part of an ellipse or an involute.

The characteristics of the rounded inwardly facing surface of the rail 16 which affect the support that it provides to a resected bone include (a) the distance (p) from the top of the rail to the point at which the rail meets the planar first surface of the transverse portion, measured parallel to the planar surface, (b) the height (q) of the rail above the first surface, measured normal to the first surface, and (c) the curvature of the surface, which can be assessed in terms of the angle (α) between the tangent to the inwardly facing surface of the rail portion at the top of the rail and the normal to the planar first surface of the transverse portion which passes through the top of the rail.

The effects of varying the characteristics of the rounded surface were investigated using a finite element analysis model of the tibial component of a knee joint prosthesis, assuming an average stress of 1.34 MPa applied to the tibial tray and a body mass of 90 kg, equivalent to a total load of 586 N applied axially to the tray. The modulus of the tibial component was assumed to be 2×10⁵ MPa and that of the cancellous bone tissue 300 MPa. The vertical stem of the tibial component was allowed to slip relative to the surrounding bone tissue, assuming a coefficient of friction which was set at 0.1 and 0.7 respectively. The low friction condition represents the condition of the prosthesis immediately after implantation, and the high friction represents the condition after some stiffening at the interface between the component and surrounding bone tissue.

The shapes of sixteen different tibial components were considered, of which one had a completely flat surface in contact with the resected tibia (as shown in FIG. 1), and fifteen had rail portions at their edges with parabolic configurations (as shown in FIGS. 2 and 3). The configurations of the rail portions varied, characterised in FIGS. 4 and 5 as piqj, where i represents the distance from the top of the rail to the point at which the rail meets the planar first surface of the transverse portion (measured parallel to the planar surface), and j represents the height of the rail above the first surface.

The parabolas are defined by the equation q=ap², where a is a variable. The values of a for various values of p and q, as well as the values of the included angle α, for the prostheses that were tested are as follows:

| Rail width | Rail height | Variable | | Inc angle |
|---|---|---|---|---|
| (p) mm | (q) mm | (a) mm⁻¹ | Gradient | (α) ° |
| 2 | 2 | 0.50 | 2.00 | 26.57 |
| 2 | 4 | 1.00 | 4.00 | 14.04 |
| 2 | 6 | 1.50 | 6.00 | 9.46 |
| 4 | 2 | 0.13 | 1.00 | 45.00 |
| 4 | 4 | 0.25 | 2.00 | 26.57 |
| 4 | 6 | 0.38 | 3.00 | 18.43 |
| 6 | 2 | 0.06 | 0.67 | 56.31 |
| 6 | 4 | 0.11 | 1.33 | 36.87 |
| 6 | 6 | 0.17 | 2.00 | 26.57 |
| 8 | 2 | 0.03 | 0.50 | 63.43 |
| 8 | 4 | 0.06 | 1.00 | 45.00 |
| 8 | 6 | 0.09 | 1.50 | 33.69 |
| 10 | 2 | 0.02 | 0.40 | 68.20 |
| 10 | 4 | 0.04 | 0.80 | 51.34 |
| 10 | 6 | 0.06 | 1.20 | 39.81 |

FIGS. 4 and 5 show the results of the finite element analysis of the degree of transverse movement of the tibial component relative to the underlying bone. When the micromotion movement is positive, the movement of the tibial component is greater than the movement of bone. When the micromotion is negative, there was more bone movement relative to movement of the tray (involving lateral expansion of the bone tissue due to hoop stresses), which occurred in particular when there was no rail on the component (as in FIG. 1). Significant positive micromotion occurred when the ratio of the distance from the top of the rail (measured parallel to the planar surface) to the point at which the rail meets the planar first surface of the transverse portion to the height of the rail above the first surface, measured normal to the first surface, was high (see p2q6).

Transverse micromotion is relatively small for certain configurations of tibial component (see p4q2, p6q2, p8q2, p10q2, p10q4).

Preparation of bone for use of such components involves resection, and preparation of a tapered cavity for receiving a peg if present. If the support formation is intended to penetrate the cortical bone tissue to provide the support, it might be preferred to prepare the bone by making indentation into which the support formation can extend. When the support formation is intended to contact the external surface of the bone (when it will preferably have a surface facing the axis of the bone which is inclined outwardly towards its free end), the outer edge of the resected bone will preferably be rounded.

1. An osteoprosthesis component which is suitable for fitting to the end of a first bone which has been resected, in a joint between that bone and a second bone, which comprises (a) a transverse portion which can be fitted across the resected end of the first bone, having a first surface which faces the resected end of the first bone when the component is implanted and a second surface for engaging the second bone directly or indirectly, in which the first surface is generally planar, and (b) a rail portion around at least part of the periphery of the component extending in a direction away from the second bone, in which the inwardly facing surface of the rail portion is generally rounded extending continuously from the planar first surface of the transverse portion when the component is viewed in cross-section.

2. An osteoprosthesis component as claimed in claim 1, in which the ratio of the distance from the top of the rail (measured parallel to the planar surface) to the point at which the rail meets the planar first surface of the transverse portion to the height of the rail above the first surface, measured normal to the first surface, is at least about 1.5, preferably at least about 1.75, more preferably at least about 2.0.

3. An osteoprosthesis component as claimed in claim 1, in which the angle between the tangent to the inwardly facing surface of the rail portion at the top of the rail and the normal to the planar first surface of the transverse portion which passes through the top of the rail is at least about 30°.

4. An osteoprosthesis component as claimed in claim 1, in which the rail portion extends around at least about 60% of the periphery of the component.

5. An osteoprosthesis component as claimed in claim 1, which is configured to be fitted to the tibia in a total knee replacement, in which the rail portion is provided on the medial and lateral edges of the component, and in which the planar first surface of the transverse portion extends to the edge of the component in at least an approximately central part of the posterior edge.

6. An osteoprosthesis component as claimed in claim 5, in which the rail portion extends continuously around the anterior, medial and lateral edges of the component, and around the medial and lateral ends of the posterior edge.

7. An osteoprosthesis component as claimed in claim 1, which includes at least one peg which depends from the transverse portion so that it can extend into the intramedullary cavity of the bone when the component is in contact with the resected end of the bone with its first surface facing the bone.
